(19) United States
(12) Patent Application Publication
(10) Pub. No.: US 2002/0031539 A1
(43) Pub. Date: Mar. 14, 2002

(54) Oxidized forms of retinoic acid as ligands for peroxisome proliferator activated receptor gamma

(75) Inventors: Jorge Plutzky, Brookline, MA (US); Ouliana Ziouzenkova, Brookline, MA (US)

Correspondence Address: PILLSBURY WINTHROP LLP, 1600 TYSONS BOULEVARD, MCLEAN, VA, 22102, US

(73) Assignee: The Brigham and Women's Hospital, Inc.

(21) Appl. No.: 09/940,858

(22) Filed: Aug. 29, 2001

Related U.S. Application Data
(60) Provisional application No. 60/228,763, filed on Aug. 30, 2000.
(60) Provisional application No. 60/246,067, filed on Nov. 7, 2000.

(51) Int. Cl. A61F 13/00; A61K 9/70; A61K 9/20

(52) U.S. Cl. 424/422; 424/434; 424/449

(57) ABSTRACT

The present invention is directed to certain oxidized forms of retinoic acid and their precursors that have been found to be effective in treating conditions that respond to agents that bind to and activate the human peroxisome proliferator activated receptor-gamma. Specific compounds include oxidized all-trans retinoic acid; oxidized 9-cis retinoic acid; and reduced 4-oxo-retinoic acid. The compounds may be used in treating a number of diseases or conditions including diabetes, low body weight, atherosclerosis, and certain forms of cancer.

### CROSS REFERENCE TO RELATED APPLICATIONS

The present application claims the benefit of U.S. provisional application No. 60/228,763, filed on Aug. 30, 2000 (pending), and No. 60/246,067, filed on Nov. 7, 2000 (pending).

### FIELD OF THE INVENTION

The present invention is directed to methods of treating patients for a variety of diseases by administering oxidized forms of retinoic acid or their esterifed analogs. Among the conditions that can be treated are diabetes, atherosclerosis and certain forms of cancer. In addition, the invention is directed to an assay method for determining whether a given test compound binds to the human peroxisome proliferator activated receptor-gamma.

### BACKGROUND OF THE INVENTION

Retinoic acid and closely related compounds have been found to have a number of important biological effects. For example, retinoic acid itself has been found to be crucial for normal embryogenesis and has been administered in clinical trials as a treatment for promyelocytic leukemia (Gudas, J. Biol. Chem. 269:15399-15402 (1994); Warrell, et al. N. Eng. J. Med. 324:1385-1393 (1991); Warrell, et al., N. Eng. J. Med. 329:177-189 (1993)). All-trans retinoic acid is also used in treating acne, psoriasis, liver spots, and skin wrinkles (Peck, et al., in: The Retinoids: Biology, Chemistry and Medicine, Sporn, et al., Raven Press, pp. 631-658 (1994); Rafai, et al., N. Eng. J. Med. 326:368-374 (1992)). The 13-cis-isomer of retinoic acid has also been used to treat dermatological conditions as well as cancer of the head, neck and cervix (Hong, et al., In: The Retinoids: Biology, Chemistry and Medicine, Roberts and Goodman, eds., Raven Press, pp. 597-630 (1994); Lippman, et al., J. Nat'l Cancer Inst. 84:241-245 (1992); Lippman, et al., J. Nat'l Cancer Inst. 84:235-241 (1992)). In addition, 4-oxo-retinol and 4-hydroxy-retinol have been suggested to be treatments for a variety of skin conditions and cancers (U.S. Pat. No. 5,962,534).

### SUMMARY OF THE INVENTION

Peroxisome proliferator activated receptors (PPARs) are located in the nucleus of cells and control the expression of specific target genes. The receptors exist in three different isoforms (PPAR-alpha, PPAR-delta, PPAR-gamma) each of which binds a different class of ligands and controls the expression of a different set of genes. PPAR-gamma has been of particular interest to pharmaceutical companies. Synthetic ligands for this receptor are “insulin sensitizers” and are in clinical use as anti-diabetic agents. It is also believed that ligands activating PPAR-gamma maybe used in the treatment of atherosclerosis and certain forms of cancer.

The present invention is based upon the discovery that certain oxidized forms of retinoic acid can bind to and activate PPAR-gamma. The compounds, which are generated by the oxidation of all-trans or 9-cis-retinoic acid and by the reduction of 4-oxo-retinoic acid, are capable of serving as therapeutic agents for all of the conditions that are responsive to PPAR-agonists. Thus, the invention is directed to methods of treating diabetes, chronically underweight patients, atherosclerosis, and cancer by administering oxidized retinoic acid, particularly oxidized forms of all trans- or 9-cis retinoic acid and reduced forms of 4-oxo-retinoic acid. One important aspect of treating atherosclerosis involves the prevention of localized atherosclerotic lesions resulting from heart transplantation. Patients may be treated with compounds both prior to and after surgery. In addition, the heart being transplanted may itself be treated with one or more forms of oxidized retinoic acid prior to the time that it is implanted. For each disease or condition treated, a therapeutically effective amount of oxidized retinoic acid is administered.

The term “therapeutically effective” means that sufficient compound is administered to a patient so that one or more symptoms associated with a disease or condition exhibit a significant improvement. For example, in diabetes, a therapeutically effective dose would lead to an increase in the amount of glucose taken up by cells in vivo or an increased sensitivity of cells to insulin. For underweight patients, therapeutic effectiveness would correspond to a dosage sufficient to promote significant weight gain and, in atherosclerosis, it would correspond to sufficient drug to reduce plaque formation. Specific cancers that may be treated with the oxidized forms of retinoic acid include breast cancer, lung cancer and liposarcomas. For these conditions, therapeutic effectiveness would correspond to a dosage sufficient to reduce tumor size, slow the proliferation of cancer cells or reduce metastasis.

In each case, it is expected that the daily dosage administered to a patient will be between 0.1 mg and 100 mg, and typically between 1 mg and 10 mg. The compounds may be administered to patients by any route and may be given either alone or in conjunction with other therapeutic agents. For example, in the treatment of diabetes, a compound may be administered in combination with insulin. The term “in combination with” means that drugs are administered in sufficiently close temporal proximity that their therapeutic effects overlap.

In another aspect, the invention is directed to a method of assaying a test compound for its ability to bind to the human PPAR-gamma receptor. This method takes advantage of the finding that certain oxidized forms of retinoic acid are ligands for the receptor. Thus, the assay method may be accomplished by incubating a source of the human PPAR-gamma receptor with the test compound and with a detectably labeled ligand selected from oxidized all-trans retinoic acid; oxidized 9-cis retinoic acid; and reduced 4-oxo-retinoic acid. By comparing the results obtained in the presence of the test compound with those from incubations carried out under essentially identical conditions but in the absence of the test compound, a determination can be made of the extent to which ligand binding has been displaced by the test compound. Many variations of this type of standard binding assay are known in the art and these variations are encompassed by the present invention.

### DETAILED DESCRIPTION OF THE INVENTION

The present invention is concerned with methods for treating diseases or conditions that have been found to be responsive to compounds that bind to and activate the PPAR-gamma receptor. Thus, the invention includes methods of treating diabetes, low body weight, atherosclerosis and certain forms of cancer. The specific compounds effective in these methods may be synthesized: 1) by the oxidation of all-trans- or 9-cis-retinoic acid (10 mM) using hydrogen peroxide (200 nM) (or t-butyl hydroperoxide) and hemin (or hemoglobin) (10 μM) at 37° C.; or 2) by the reduction of 4-oxo-retinoic acid (10 mM) using sodium borohydride (10fold molar excess). Lipophylic products can be extracted using a standard water/ethanol/hexan (1/1/5, v/v) extraction procedure. All-trans and 9-cis retinoic acid are available commercially and 4-oxo-retinoic acid may be synthesized using methods that are well-known in the art (Samokyszyn, et al., J. Biol. Chem. 275(10):690806914 (2000)). Other specific oxidized retinoic acids that could potentially be used in the methods disclosed herein but which are not presently thought to be preferred are 18-hydroxy-retinoic acid; 4-hydroxy-retinoic acid; 4-oxo-retinoic acid; and 3,4-didehydro-retinoic acid. In addition, cellular precursors of these oxidized retinoic acids that could potentially be used include: 8′-apo-carotenal; and 14′-apo-carotenal. 4-oxo-retinoic acid and 3,4-didehydro-retinoic acid. It will be understood that the invention includes not only the use of the compounds specifically named but also forms that have been modified in a manner that is routine and well known in the art. For example, the compounds may be chemically stabilized by methylation or acetylation.

The total dosage of compound to be administered to a patient should be at least the amount required to reduce or eliminate symptoms associated with the disease being treated. For example, a patient being treated for a neoplastic disease should be given sufficient compound to retard or reverse abnormal cellular growth. Similarly, patients being treated for diabetes should show increased insulin responsiveness, i.e. increased cellular glucose uptake in response to insulin.

Physicians may begin by administering relatively small doses of compound and then adjust the dosage upward as it becomes clear that a patient can tolerate the treatment. For example, a physician may begin by administering 0.1 mg per day and then increase dosage up to 100 mg per day or higher using changes in disease symptoms as a guide. The final dosage may be provided in either a single or multiple regimen with the latter being generally preferred. These are simply guidelines since the actual dose must be carefully selected and titrated by the attending physician based upon clinical factors unique to each patient. The optimal daily dose will be determined by methods known in the art and will be influenced by factors such as the age of the patient, the disease state, side effects associated with the particular agent being administered and other clinically relevant factors. In some cases, a patient may already be taking medications at the time that treatment with one of the oxidized forms of retinoic acid is initiated. These other medications may be continued during the time that the retinoic acid compound is being administered provided that no adverse side effects are reported by the patient.

The present invention is not limited to any particular dosage form or route of administration. Although oral administration will generally be most convenient, the invention is compatible with parenteral, transdermal, sublingual, buccal, or implantable forms of administration as well. Compounds may be given in a substantially purified form, or as part of a pharmaceutical composition containing one or more excipients or flavoring agents. Compositions may also include other active agents for the treatment of patients. The preparations may be solid or liquid and take any of the pharmaceutical forms presently used in medicine, e.g., tablets, gel capsules, granules, suppositories, transdermal compositions or injectable preparations. To retain activity, the oxidized retinoic acid compounds should, preferably, be stored in nitrogen or argon and be protected from light.

The active compound, or compounds, may be incorporated into dosage forms in conjunction with the vehicles that are commonly employed in pharmaceutical preparations, e.g. talc, gum arabic, lactose, starch, magnesium stearate, cocoa butter, aqueous or non-aqueous solvents, paraffin derivatives, glycols, etc. Methods for preparing appropriate formulations are well-known in the art (see e.g., Remington's Pharmaceutical Sciences, 16th ed., A. Oslo ed., Easton, Pa. (1980)).

In general, it is expected that patients will be treated with an oxidized retinoic acid compound administered at a daily dosage in the range of 0.1 to 100 mg. In order to determine the effect of treatment on disease, patients should be evaluated on a regular basis over an extended period of time. It may take several weeks for the full therapeutic effect of a treatment to become apparent. Standard clinical methods may be used for assessing the extent to which an improvement in one or more symptoms associated with the disease has been achieved by drug administration.

The invention is also directed to an improved method for assaying a test compound to determine its binding affinity for the human PPAR-gamma receptor (see, Kliewer, et al., Cell 83:813-819 (1995) and Nagy, et al., Cell 93:229-240 (1998)). The essential feature of this method is that recombinant PPAR-gamma ligand binding domain is incubated together with an oxidized form of retinoic acid and with the compound being tested for binding activity. The preferred source of receptor is a cell that has been recombinantly transformed to express human PPAR-gamma ligand binding domain. After binding is complete, the receptor is separated from the solution containing the oxidized retinoic acid ligand and test compound, e.g. by size exclusion chromatography, and the amount of binding that has occurred is determined.

Preferably, the oxidized retinoic acid used as a ligand in the assay is detectably labeled. Any of the compounds that have been used for detecting ligands in binding assays are compatible with the present method including radiosotopes and fluorescent or chemiluminescent labels. Among the most commonly used fluorescent labeling compounds are fluorescein isothiocyanate, rhodamine, phycoerythrin, phycocyanian, allophycocyanin, ophthaldehyde and fluorescamine. Useful chemiluminescent compounds include luminol, isoluminol, theromatic acridinium ester, imidazole, acridinium salt, oxalate ester.

Nonspecific binding may be determined by carrying out the binding reaction in the presence of a large access of unlabeled ligand. For example, isotopically labeled and oxidized retinoic acid may be incubated with receptor in the presence of a thousandfold access of unlabeled oxidized acid. Nonspecific binding should be subtracted from total binding, i.e. binding in the absence of unlabeled ligand, to arrive at the specific binding for each sample tested. Other steps such as washing, stirring, shaking, filtering and the like may be included in the assays as necessary. The specific binding obtained in the presence of test compound is compared with that obtained in the presence of labeled ligand alone to determine the extent to which the test compound has itself bound to PPAR-gamma.

In performing binding assays, care must be taken to avoid artifacts which may make it appear that a test compound is interacting with the PPAR-gamma receptor when, in fact, binding is being inhibited by some other mechanism. For example, the compound being tested should be in a buffer which does not itself substantially inhibit binding and should, preferably, be tested at several different concentrations. It is also preferable that compounds identified as displacing the binding of ligand to receptor be reexamined in a concentration range sufficient to perform a Scatchard analysis on results. This type of analysis is well-known in the art and can be used for determining the affinity of a test compound for receptor (see, e.g. Ausubel, et al., Current Protocols in Molecular Biology, 11.2.1-11.2.19 (1993)). Computer programs may be used to help in the analysis of results (See e.g., Munson, P. Method Enzymol. 92:543-577 (1983)).

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

What is claimed is:

1. A method of treating a patient for diabetes comprising: administering a therapeutically effective amount of a compound selected from the group consisting of: oxidized all-trans retinoic acid; oxidized 9-cis retinoic acid; and reduced 4-oxo-retinoic acid.

2. The method of claim 1, wherein said compound is administered in combination with insulin.

3. The method of claim 1, wherein said compound is administered to said patient at a daily dose of between 0.1 mg and 100 mg.

4. The method of claim 1, wherein said compound is oxidized all-trans retinoic acid.

5. The method of claim 1, wherein said compound is oxidized 9-cis retinoic acid.

6. The method of claim 1, wherein said compound is reduced 4-oxo-retinoic acid.

7. A method of treating a patient for being underweight comprising: administering to said patient a therapeutically effective amount of a compound selected from the group consisting of: oxidized all-trans retinoic acid; oxidized 9-cis retinoic acid; and reduced 4-oxo-retinoic acid.

8. The method of claim 7, wherein said compound is administered to said patient at a daily dose of between 0.1 mg and 100 mg.

9. The method of claim 7, wherein said compound is oxidized all-trans retinoic acid.

10. The method of claim 7, wherein said compound is oxidized 9-cis retinoic acid.

11. The method of claim 7, wherein said compound is reduced 4-oxo-retinoic acid.

12. A method of treating a patient for atherosclerosis comprising: administering a therapeutically effective amount of a compound selected from the group consisting of: oxidized all-trans retinoic acid; oxidized 9-cis retinoic acid; and reduced 4-oxo-retinoic acid.

13. The method of claims 12, wherein said compound is administered to said patient at a daily dose of between 0.1 mg and 100 mg.

14. The method of claim 12, wherein said compound is oxidized all-trans retinoic acid.

15. The method of claim 12, wherein said compound is oxidized 9-cis retinoic acid.

16. The method of claim 12, wherein said compound is reduced 4-oxo-retinoic acid.

17. A method of treating a patient for cancer comprising: administering a therapeutically effective amount of a compound selected from the group consisting of: oxidized all-trans retinoic acid; oxidized 9-cis retinoic acid; and reduced 4-oxo-retinoic acid.

18. The method of claim 17, wherein said cancer is breast cancer; lung cancer; or a liposarcoma.

19. The method of claim 17, wherein said compound is administered to said patient at a daily dose of between 0.1 mg and 100 mg.

20. The method of claim 17, wherein said compound is oxidized all-trans retinoic acid.

21. The method of claim 17, wherein said compound is oxidized 9-cis retinoic acid.

22. The method of claim 17, wherein said compound is reduced 4-oxo-retinoic acid.

23. A method of assaying a test compound for its ability to bind to the human PPAR-gamma receptor, comprising:
a) incubating recombinant PPAR-gamma binding domain with said test compound and with a ligand selected from the group consisting of:
oxidized all-trans retinoic acid; oxidized 9-cis retinoic acid; and reduced 4-oxo-retinoic acid; and
b) determining the extent to which the binding of said ligand is displaced by said test compound.
